(19) United States
(12) Patent Application Publication
(10) Pub. No.: US 2017/0281561 A1
(43) Pub. Date: Oct. 5, 2017

(54) DRUG DELIVERY FABRIC HAVING DRUG-CONTAINING LAYER

(71) Applicant: VENTEX CO.,LTD., SEOUL (KR)

(72) Inventors: KYUNG CHAN GO, Gyeonggi-Do (KR); GWANG WUNG YANG, Gyeonggi-Do (KR); YONG HWAN RHO, Incheon (KR)

(21) Appl. No.: 15/507,644

(22) PCT Filed: Aug. 28, 2014
(86) PCT No.: PCT/KR2014/008025

§ 371 Date: Feb. 28, 2017

(30) Foreign Application Priority Data
Aug. 28, 2014 (KR) 10-2014-0113153

(51) Int. Cl. A61K 9/70; A61K 9/14; A61K 9/50

(52) U.S. Cl. CPC A61K 9/703; A61K 9/50; A61K 9/14; A61K 9/7061

(57) ABSTRACT

The present invention relates to a drug delivery fabric having a drug-containing layer, wherein the drug delivery fabric comprises a surface layer, a middle layer, and a drug-containing layer, the middle layer is formed of a yarn having hydrophilicity relatively equal to or higher than that of the surface layer, the drug-containing layer is formed of a cellulose derivative film containing a drug.

### TECHNICAL FIELD

The present invention relates to a fabric having a drug delivery function, and more particularly to a drug delivery fabric having a drug-containing layer in which the drug is transferred to the body in one direction to be absorbed in the body well.

### BACKGROUND ART

As the most common method of administering a drug to the human body, bolus injection and oral administration methods are included. The bolus injection is to directly inject the drug into the vein and rapidly acts on the entire body through the vein-heart-artery. Oral administration is to administering orally drugs in forms of liquids, powders, tablets, capsules, and the like, and the drug may rapidly reach a therapeutically effective blood concentration depending on the degree of absorption by the gastrointestinal circulation.

Most of drugs through the percutaneous are absorbed between cells and cells, through the cells, and through skin appendages such as the sweat gland, and the drug emitted from a percutaneous formulation is distributed to the whole body after being absorbed to the dermis through a living epidermis and from the dermis to the carpillary through a principle of diffusing and distributing to the stratum corneum. A method using the percutaneous absorption largely includes a patch, iontophoresis, sonophoresis, a microneedle, and the like.

The drug delivery through the percutaneous maintains the blood concentration for an administration period by injection or oral administration to remove a valley-peak phenomenon of a blood drug concentration which is frequently shown in injections and there are almost no side effects including gastrointestinal disorders, liver disorders, and the like which may be caused by the oral drugs. Further, in the case of the injection, when it is considered that a rapid increase in blood concentration shown immediately after drug administration has a toxic risk, there is an advantage in that a percutaneous delivery system is further advantageous in terms of safety or efficiency.

However, when the drug needs to be constantly transferred for a long time, generally, the percutaneous delivery system is not considered as a useful method for reaching a high blood concentration of the drug in a short time. The reason is that most of percutaneous drug delivery apparatuses has a considerable delay time (a time taken to reach an effective blood concentration of the drug after administration) to obtain a required or desired blood concentration after being attached to the skin.

Korean Patent Registration No. 1051977 relates to “an apparatus having a thin battery and an iontophoresis patch”, and there is provided an apparatus such as an iontophoresis patch having a thin battery in which a battery and a device connected thereto are integrated, a current density is improved, and it is advantageous to improve slimness and manufacturing productivity. Korean Patent Registration No. 950584 relates to “a battery integrated iontophoresis patch”, and there is provided au iontophoresis patch, which can improve manufacturing productivity by integrating the iontophoresis patch and a battery, achieve high efficiency by minimizing contact resistance with the skin, and apply an interest substance having weak electrochemical resistance by preventing the interest substance of the iontophoresis patch from being broken by participating in an electrode reaction.

As such, the percutaneous drug transfer system is the most used as the patch, but there are disadvantages in that the patch is used for a single use and thus continuous consumption is required, and the patch is relatively expensive to be used repeatedly and thus cannot be used for a long time. Further, the iontophoresis, sonophoresis, and the microneedle are machines in which prices are also high, the help of experts is required at the time of use, and portability is deteriorated, and there is a disadvantage in that the machines cannot be conveniently used anytime and anywhere, and thus a novel percutaneous drug transfer system capable of being continuously used, having excellent portability, and being conveniently used regardless of time and space is required.

### DISCLOSURE

### Technical Problem

The present invention is developed for solving the problems in the related art, and an object of the present invention is to provide a fabric having a drug delivery function capable of continuously transferring a drug to the skin by using the fabric which contains the drug and allows moisture to be transferred in one direction.

Further, another object of the present invention is to provide a fabric having a drug delivery function capable of preventing moisture from being discharged to the outside so that the skin may be hydrated to facilitate percutaneous delivery of a drug.

Further, yet another object of the present invention is to provide a fabric having a drug delivery function capable of preventing the skin from being damaged by a characteristic of the fabric formed of fibers.

### Technical Solution

An aspect of the present invention provides a drug delivery fabric having a drug-containing layer, wherein the drug delivery fabric includes a surface layer, a middle layer, and a drug-containing layer, the middle layer is formed of a yarn having hydrophilicity relatively equal to or higher than that of the surface layer, the drug-containing layer is formed of a cellulose derivative film containing a drug.

Further, the middle layer may have tissue density relatively higher than that of the surface layer.

Further, the surface layer may be formed of any one of polypropylene-based fibers, polyester-based fibers, polyamide-based fibers, and cellulose-based fibers or by blending two or more fibers thereof.

Further, the middle layer may be formed of any one of polyamide-based fibers, polyacryl-based fibers, polyester-based fibers, cellulose-based fibers, and protein-based fibers or by blending two or more fibers thereof.

Further, the drug-containing layer may be a protein-based film or a cellulose derivative film.

Further, the drug contained in the drug-containing layer may be included by encapsulation.

Further, the drug contained in the drug-containing layer may be caffeine.

Another aspect of the present invention provides clothes having a drug delivery function including the drug delivery fabric.

### Advantageous Effects

According to the present invention, it is possible to transfer continuously the drug contained in the fabric to the skin by the drug delivery having the drug-containing layer of the present invention.

Further, it is possible to increase a diffusion coefficient by hydrating the skin through the fabric having high moisture and improve a penetration speed of the drug by increasing the concentration of the drug which is in contact with the skin.

Further, it is possible to enhance a drug effect by preventing the drug transferred to the skin from being transferred to the outside of the fabric again so as not to consume the used drug and have an economical effect because there is no consumed drug.

### DESCRIPTION OF DRAWINGS

FIG. 1 is a cross-sectional view illustrating a drug delivery fabric having a drug-containing layer of the present invention.

FIG. 2 is a diagram of an exemplary embodiment of clothes manufactured by a fabric having a drug delivery function of the present invention.

FIG. 3 is a diagram illustrating a Franz diffusion cell capable of testing percutaneous absorption.

### DESCRIPTION OF MAIN REFERENCE NUMERALS OF DRAWINGS

### Best Mode

Hereinafter, preferred exemplary embodiments of the present invention will be described in detail with reference to the accompanying drawings. First, it should be noted that like reference numerals designate like constituent elements or components in the drawings. In describing the present invention, a detailed description of publicly known functions or configurations incorporated herein will be omitted so as not to make the subject matter of the present invention unclear.

The terms representing the degree used in this specification of ‘approximately’, ‘substantially’, and the like are used as the value or a meaning close to the value when unique manufacturing and material tolerances are proposed in the aforementioned meaning, and used for preventing the disclosed content in which accurate or absolute figures are mentioned in order to help in the understanding of the present invention from being wrongly used by unscrupulous infringers.

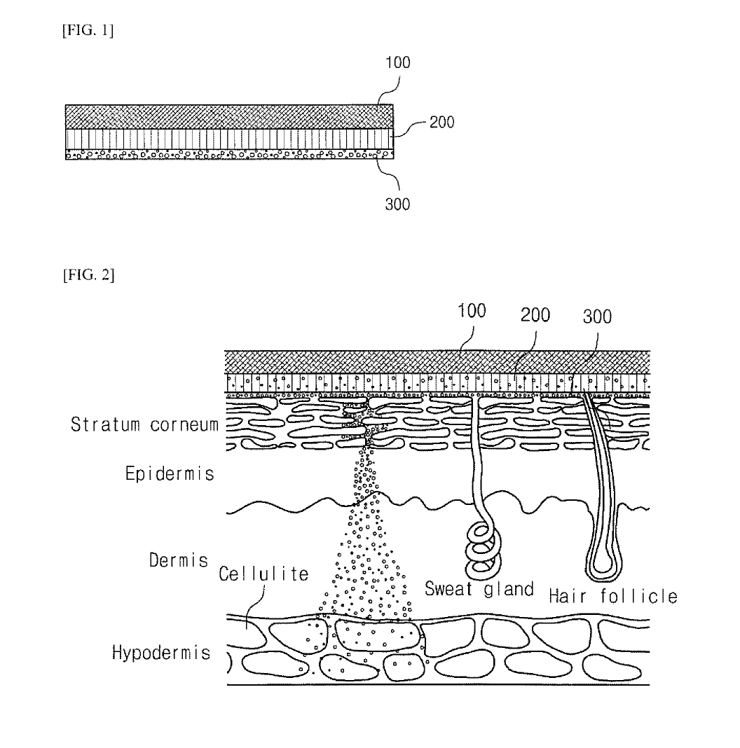

FIG. 1 is a cross-sectional view illustrating a drug delivery fabric having a drug-containing layer of the present invention and FIG. 2 is a diagram of an exemplary embodiment of clothes manufactured by a fabric having a drug delivery function of the present invention.

The present invention relates to a drug delivery fabric having a drug-containing layer, and as illustrated in FIG. 1, the fabric is a drug delivery fabric which is formed by a surface layer 100 that prevents the drug from being transferred to the outside of the skin, a middle layer 200 that transfers the drug toward the skin, and a drug-containing layer 300 that is formed of a cellulose derivative film containing the drug.

The middle layer 200 is formed of a yarn having hydrophilicity relatively equal to or higher than that of the surface layer 100 to prevent moisture from being transferred to the surface layer 100 from the middle layer 200 and needs to be formed to transfer the moisture to the middle layer 200 from the surface layer 100.

The surface layer 100 may be formed of any one of polypropylene-based fibers, polyester-based fibers, polyamide-based fibers, and cellulose-based fibers or by blending two or more fibers thereof. The middle layer 200 may be formed of any one of polyamide-based fibers, polyacryl-based fibers, polyester-based fibers, cellulose-based fibers, and protein-based fibers or by blending two or more fibers thereof. The surface layer 100 needs to be constituted to prevent hydrophilicity from being higher than that of the middle layer 200.

For moisture transfer in one direction between the surface layer 100 and the middle layer 200, the middle layer 200 may be formed to have tissue density relatively higher than that of the surface layer 100.

The surface layer 100 and the middle layer 200 are formed of respective fabrics and then may be formed by a laminating method and the like and may be formed of a double-woven fabric through weaving or knitting.

As such, when the drug delivery fabric is constituted by the middle layer 200 having the relatively high hydrophilicity and the high tissue density and the surface layer, by a capillary phenomenon due to a difference in tissue density between the surface layer 100 and the middle layer 200, the moisture or the drug absorbed in the surface layer 100 is transferred to the middle layer 200 having the high density and the high hydrophilicity.

The surface layer 100 and the middle layer 200 may be formed by further containing an elastic yarn in order to enhance skin adhesion. The elastic yarn may use polyurethane-based or copolymer polyester-based fibers.

The drug-containing layer 300 is formed of a cellulose derivative film containing the drug.

The cellulose derivative film is a film made of a water-soluble polymer called cellulose ether.

The cellulose may be made of an insoluble natural polymer having crystallinity of 30 to 65% due to intermolecular hydrogen bonding or a water-soluble polymer called cellulose ether through etherification.

The cellulose ether may include methylcellulose (MC), ethylcellulose (EC), hydroxyethylcellulose (HEC), hydroxyethylmethylcellulose (HEMC), hydroxypropylmethylcellulose (HPMC), hydroxypropylcellulose (HPC), microcrystallin cellulose (MCC), carboxymethylcellulose (CMC), and the like, and the cellulose derivative film of the present invention may be manufactured by using the cellulose ether.

The cellulose derivative film containing the drug of the present invention may be manufactured by containing the drug when forming the film using the cellulose ether.

As one example, the film may be manufactured by a solvent casting method in which the drug is directly dissolved in a solution of 3 to 10% of cellulose ether such as methylcellulose, carboxymethylcellulose, and hydroxypropylmethylcellulose and then glycerol as a plasticizer is added. Alternatively, the film may be manufactured by a phase-inversion method in which the drug is dissolved in a solution of 5 to 15% of cellulose ether.

As another example, the film may be manufactured by a film type nanoweb by containing the drug in cellulose ether and nano-fiberizing through electro-spinning.

The content of the drug contained in the cellulose derivative film needs to be adjusted according to a used drug and may be contained with 0.1 to 10 wt % of the weight of the cellulose derivative film for strength or ductility of the cellulose derivative film.

Further, the drug contained in the cellulose derivative film may be decomposed at the time of manufacturing the film and thus the drug may be encapsulated to prevent the drug from being decomposed and then included in the film.

The encapsulation of the drug may be performed by general micro-encapsulation of the drug using a capsule wall material such as calcium carbonate, silk, collagen, cellulose, chitosan, gelatin, and casein phthalic anhydride or encapsulation of injecting the drug into porous particles such as silica, titanium oxide, and zeolite.

The drug may use any drug suitable for percutaneous absorption and may use caffeine having functions of decomposing cellulite composed of fat and fiber and decomposing body fat (visceral fat and subcutaneous fat).

By the drug delivery fabric having the drug-containing layer according to the present invention which is formed above, most of drugs through the percutaneous are absorbed through a space between the cells of the stratum corneum as illustrated in FIG. 2 and absorbed to the dermis through a living epidermis and from the dermis to the carpillary by a principle of being diffused and distributed through the stratum corneum.

After the drugs pass through the stratum corneum, the drugs are easily diffused and distributed because there is no material that inhibits the diffusion of the drugs, and thus in the drug delivery through the percutaneous, it is most important that the drugs rapidly pass through the stratum corneum.

As such, a speed at which the drug is percutaneously delivered is as illustrated in Equation 1 below as a percutaneous absorption kinetics.

$$J = DK \frac{C}{h} = PC \qquad [ Equation 1 ]$$

(K: Fat-soluble degree of drug, D: Diffusion coefficient, C: Drug concentration, h: Skin thickness)

The fat-soluble degree K of drug in the percutaneous absorption kinetics is a unique characteristic of the used drug and the skin thickness h is a unique characteristic of each person, and thus it is not easy to adjust the characteristics with the fabric.

The diffusion coefficient D is changed depending on hydration of the skin and a temperature, and as the skin is hydrated, the diffusion coefficient D increases as the temperature increases.

In the present invention, the drug-containing layer 300 made of the water-soluble polymer is in contact with the skin as illustrated in FIG. 2 and serves to hydrate the skin, promote the hydration of the skin by configuring the surface layer 100 and the middle layer 200 which transfer the moisture to the skin, and increase the diffusion coefficient D by adjusting the concentration.

Further, the drug-containing layer 300 prevents the drug from being transferred to the surface layer of the fabric to increase the concentration C of the drug which is in contact with the skin.

As such, the fabric having the drug delivery function of the present invention allows the drug to be delivered into the inside of the percutaneous by improving the percutaneous speed due to the increase in the diffusion coefficient D and the increase in concentration C of the drug in contact with the skin through the skin hydration.

Hereinafter, Examples of a method for manufacturing a drug delivery fabric having a drug-containing layer according to the present invention will be described, but the present invention is not limited to the Examples.

### EXAMPLES 1 TO 4

A double-woven knitted fabric was manufactured by a fabric composition illustrated in Table 1 below to manufacture the surface layer 100 and the middle layer 200, a hydroxypropyl methylcellulose (HPMC) film was used as the drug-containing layer 300, and caffeine was used as the drug. The caffeine was contained in the film by adding 5% of a polymer weight when the film was manufactured.

The knitted fabric of the surface layer and the middle layer and the drug-containing layer are bonded to each other through a laminating method to manufacture the drug delivery fabric having the drug-containing layer of the present invention.

TABLE 1

| | | | Weight |
|---|---|---|---|
| Classification | | Fiber type | ratio |
| Example 1 | Surface layer | Polypropylene fiber | 37% |
| | Middle layer | Modal fiber | 63% |
| Example 2 | Surface layer | Polyester fiber/cotton blended yarn | 50% |
| | Middle layer | Polyester fiber/cotton blended yarn | 50% |
| Example 3 | Surface layer | Polypropylene fiber | 41.30% |
| | Middle layer | Modal fiber | 58.70% |
| Example 4 | Surface layer | Polyester fiber | 41.50% |
| | Middle layer | Modal fiber | 58.50% |

Percutaneous Absorption Test

A percutaneous absorption test of the drug delivery fabric of Examples 1 to 4 manufactured above was performed. As Comparative Example, a caffeine-containing hydroxypropylmethylcellulose film manufactured in the Examples was used.

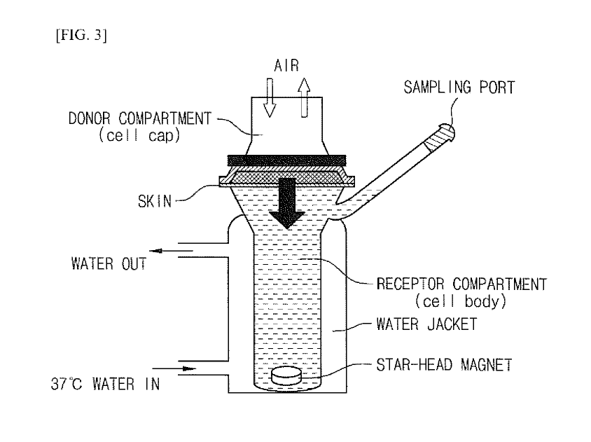

The percutaneous absorption test was performed by using a Franz diffusion Cell which may test percutaneous absorption of FIG. 3 and PBS saline was used as a receptor chamber.

Results of the percutaneous absorption test in Examples 1 to 4 and Comparative Example were in Table 2.

TABLE 2

| Classification | 0H | 2H | 4H | 6H | 8H | 10H | 12H |
|---|---|---|---|---|---|---|---|
| Example 1 | 0 | 58.2 | 104.0 | 156.6 | 172.3 | 186.0 | 228.1 |
| Example 2 | 0 | 21.6 | 34.0 | 54.6 | 62.6 | 64.0 | 64.6 |
| Example 3 | 0 | 35.1 | 60.6 | 97.1 | 112.1 | 125.1 | 144.5 |
| Example 4 | 0 | 21 | 29.8 | 48.8 | 63.5 | 67.1 | 69.2 |
| Comparative Example | 0 | 9.9 | 18.1 | 29.7 | 39.6 | 42.9 | 47.8 |

As illustrated in Table 2, as compared with the case of using only the HPMC film in Comparative Example, in Example which was the drug delivery fabric having the drug-containing layer of the present invention, it can be seen that a percutaneous absorption rate is excellent as at least 50% or more, and in the case of Example 1, it can be seen that the percutaneous absorption rate is 5 times larger than that of Comparative Example.

Therefore, in the drug delivery fabric having the drug-containing layer of the present invention, it can be seen that the drug delivery function through the percutaneous is very excellent.

1. A drug delivery fabric having a drug-containing layer, wherein the drug delivery fabric includes a surface layer, a middle layer, and a drug-containing layer, the middle layer is formed of a yarn having hydrophilicity relatively equal to or higher than that of the surface layer, and the drug-containing layer is formed of a cellulose derivative film containing a drug.

2. The drug delivery fabric having a drug-containing layer of claim 1, wherein the middle layer has tissue density relatively higher than that of the surface layer.

3. The drug delivery fabric having a drug-containing layer of claim 1, wherein the surface layer is formed of any one of polypropylene-based fibers, polyester-based fibers, polyamide-based fibers, and cellulose-based fibers or by blending two or more fibers thereof.

4. The drug delivery fabric having a drug-containing layer of claim 1, wherein the middle layer is formed of any one of polyamide-based fibers, polyacryl-based fibers, polyester-based fibers, cellulose-based fibers, and protein-based fibers or by blending two or more fibers thereof.

5. The drug delivery fabric having a drug-containing layer of claim 1, wherein the drug contained in the drug-containing layer is included by encapsulation.

6. The drug delivery fabric having a drug-containing layer of claim 1, wherein the drug contained in the drug-containing layer is caffeine.

7. Clothes having a drug delivery function comprising the drug delivery fabric of any one of claims 1.

8. Clothes having a drug delivery function comprising the drug delivery fabric of claim 2.

9. Clothes having a drug delivery function comprising the drug delivery fabric of claim 3.

10. Clothes having a drug delivery function comprising the drug delivery fabric of claim 4.

11. Clothes having a drug delivery function comprising the drug delivery fabric of claim 5.

12. Clothes having a drug delivery function comprising the drug delivery fabric of claim 6.
